(19) United States
(12) Patent Application Publication
(10) Pub. No.: US 2004/0067236 A1
(43) Pub. Date: Apr. 8, 2004

(54) Immunogenic compositions comprising liver stage malarial antigens

(76) Inventors: Joe Cohen, Rixensart (BE); Pierre Druilhe, Paris (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,253

(22) PCT Filed: Oct. 23, 2001
(86) PCT No.: PCT/EP01/12349

(30) Foreign Application Priority Data
Oct. 25, 2000 (EP) 00203724.0

(51) Int. Cl. A61K 39/00; A61K 31/739; C07K 14/44

(52) U.S. Cl. 424/185.1; 514/54; 530/350

(57) ABSTRACT

A vaccine composition comprising a Th1-inducing adjuvant in combination with a protecting Liver Stage Antigen or immunological fragment thereof of a human malaria parasite, especially Plasmodium falciparum, with the proviso that when the immunological fragment is an immunological fragment of LSA-3 the Th1-inducing adjuvant is not Montanide. In one preferred aspect the Th1-inducing adjuvant comprises QS21, De-O-acylated monophosphoryl lipid A (3D-MPL) and an oil in water emulsion wherein the oil in water emulsion has the following composition: a metabolisible oil, such a squalene, alpha tocopherol and tween 80. In a further preferred aspect the protecting Liver Stage Antigen is Liver Stage Antigen 3 (LSA-3) or an immunological fragment thereof. A multivalent vaccine composition is also provided comprising the vaccine composition of the invention and in addition at least one other protecting antigen or an immunological fragment thereof, of a malaria parasite.

The present invention relates to novel vaccine formulations, to methods of their production and to their use in medicine. In particular, the present invention relates to a malaria antigen known as Liver Stage Antigen 3, or an immunological fragment thereof, in association with a Th-1 inducing adjuvant such as an oil in water emulsion or a vesicular adjuvant formulation comprising cholesterol, a saponin and optionally a lipopolysaccharide derivative. These and other aspects of the invention are described hereinbelow.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosarnine, has been described by Ribi et al (1986, Immunology and Immunopharmacology of bacterial endotoxins, Plenum Publ. Corp., NY, p407-419).

A farther detoxified version of MPL results from the removal of the acyl chain from the 3-position of the disaccharide backbone, and is called 3-O-Deacylated monophosphoryl lipid A (3D-MPL). 3 De-O-acylated monophosphoryl lipid A is known from GB2 220 211 (Ribi). Chemically it is a mixture of 3 De-O-acylated monophosphoryl lipid A with 4, 5 or 6 acylated chains and is manufactured by Ribi Immunochem Montana. GB 2122204B also discloses the preparation of diphosphoryl lipid A, and 3-O-deacylated variants thereof. Other purified and synthetic lipopolysaccharides have been described (U.S. Pat. No. 6,005,099 and EP 0 729 473 B1; Hilgers et al., 1986, Int.Arch.Allergy.Immunol., 79(4):392-6; Hilgers et al., 1987, Immunology, 60(1):141-6; and EP 0 549 074 B1).

A preferred form of 3 De-O-acylated monophosphoryl lipid A (3D-MPL) is in the form of an emulsion having a small particle size less than 0.2 μm in diameter, disclosed in International Patent Application No. WO 92/116556 (SmithKline Beecham Biologicals s.a.). See also WO 94/21292.

Aqueous formulations comprising monophosphoryl lipid A and a surfactant have been described in WO98/43670A2.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemholytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of theirproduction is disclosed in U.S. Pat. No. 5,057,540 and EP 0 362 279 B 1. Other saponins which have been used in systemic vaccination studies include those derived from other plant species such as Gypsophila and Saponaria (Bomford et al., Vaccine, 10(9):572-577, 1992).

QS21 is a Hplc purified non toxic fraction of a saponin from the bark of the South American tree Quillaja Saponaria Molina and its method of its production is disclosed (as QA21) in U.S. Pat. No. 5,057,540.

Oil emulsion adjuvants have been known for many years, including work on Freund's complete and incomplete mineral oil emulsion adjuvants. Since that time much work has been performed to design stable and well tolerated alternatives to these potent, but reactogenic, adjuvant formulations.

Many single or multiphase emulsion systems have been described. Oil in water emulsion adjuvants per se have been suggested to be useful as adjuvant compositions (EP 0 399 843B), also combinations of oil in water emulsions and other active agents have been described as adjuvants for vaccines (WO 95/17210). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

In order for any oil in water composition to be suitable for human administration, the oil phase of the emulsion system preferably comprises a metabolisable oil. The meaning of the term metabolisable oil is well known in the art. Metabolisable can be defined as “being capable of being transformed by metabolism” (Dorland's Illustrated Medical Dictionary, W. B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil virtue of the fact that it is an intermediate in the biosynthesis of cholesterol (Merck index, 10th Edition, entry no.8619).

The oil in water emulsions which form part of the present invention when formulated with 3 D-MPL and QS21 are preferential stimulators of IgG2a production and TH1 cell response. This is advantageous, because of the known implication of TH₁ response in cell mediated response. Indeed in mice induction of IgG2a is correlated with such an immune response.

The observation that it is possible to induce strong cytolytic T lymphocyte responses is significant as these responses, in certain animal models have been shown to induce protection against disease.

The present inventors have shown that the combination of the adjuvants QS21 and 3D-MPL together with an oil in water emulsion with an antigen results in a powerful induction of CS protein specific CTL in the spleen. QS21 also enhances induction of CTL on its own, while 3D-MPL does not.

Induction of CTh is easily seen when the target antigen is synthesised intracellularly (e.g. in infections by viruses, intracellular bacteria, or in tumours), because peptides generated by proteolytic breakdown of the antigen can enter the appropriate processing pathway, leading to presentation in association with class I molecules on the cell membrane. However, in general, pre-formed soluble antigen does not reach this processing and presentation pathway, and does not elicit class I restricted CTL. Therefore conventional non-living vaccines, while eliciting antibody and T helper responses, do not generally induce CTL mediated Immunity. The combination of the two adjuvants QS21 and 3D-MPL together with an oil in water emulsion can overcome this serious limitation of vaccines based or recombinant proteins, and induce a wider spectrum of immune responses.

CTL specific for CS protein have been shown to protect from malaria in mouse model systems (Romero et al. Nature 341:323 (1989)). In human trials where volunteers were immunised using irradiated sporozoites of P. falciparum, and shown to be protected against subsequent malaria challenge, induction of CTL specific for CS epitopes was demonstrated (Malik et al. Proc. Natl. Acad. Sci. USA 88:3300 (1991)).

The ability to induce CTL specific for an antigen administered as a recombinant molecules is relevant to malaria vaccine development, since the use of irradiated sporozoites would be impractical, on the grounds of production and the nature of the immune response.

In certain systems, the combination of 3D-MPL and QS21 together with an oil in water emulsion have been able to synergistically enhance interferon γ production.

IFN-γ secretion is associated with protective responses against intracellular pathogens, including parasites, bacteria and viruses. Activation of macrophages by IFN-γ enhances intracellular killing of microbes and increases expression of Fc receptors. Direct cytotoxicity may also occur, especially in synergism with lymphotoxin (another product of TH1 cells). IFN-γ is also both an inducer and a product of NK cells, which are major innate effectors of protection. TH1 type responses, either through IFN-γ or other mechanisms, provide preferential help for IgG2a immunoglobulin isotypes.

Particularly preferred adjuvants which may be used in the invention described herein are combinations of 3D-MPL and QS21 (EP 0 671 948 B1), oil in water emulsions comprising 3D-MPL and QS21 (WO 95/17210, PCT/EP98/05714), 3D-MPL formulated with other carriers (EP 0 689 454 B1), or QS21 formulated in cholesterol containing liposomes (WO 96/33739), or immunostimulatory oligonucleotides (WO 96/02555).

RTS is a hybrid protein comprising substantially all the C-terminal portion of the circumsporozoite (CS) protein of P.falciparum linked via four amino acids of the preS₂ portion of Hepatitis B surface antigen to the surface (S) antigen of hepatitis B virus (HBV). The structure of RTS and the molecules from which it is derived is disclosed in International Patent Application Publication Number WO 93/10152.

When expressed in yeast RTS is produced as a lipoprotein particle, and when it is co-expressed with the S antigen from HBV it produces a mixed particle known as RTS,S.

Liver Stage Antigens are described in Malaria, Parasite Biology, Pathogenesis and Protection (1998 ASM Press, Washington D.C., edited by Irwin W. Sherman), especially Chapter 34 (P. Druilhe et al.).

A 26-amino acid synthetic peptide based on Plasmodium falciparum liver stage antigen 3 (LSA-3) is described in Eur J. Immunol., 1997, 27, 1242-1253 (L. BenMohamed et al).

The immunogenicity of 12 synthetic peptides derived from four new Plasmodium falciparum molecules expressed at pre-erythrocytic stages of the human malaria parasite was reported in Vaccine 18 (2000), pages 2843-2855 (L BenMohamed et al).

In these studies the adjuvant Montanide ISA-51 (SEPPIC, Quai D'Orsay, France) was used. There is no report, however, of such peptides being combined with other adjuvants. The present invention is based on the surprising discovery that a Th-1 inducing adjuvant especially an oil in water emulsion which preferably comprises tocopherol, as such or in combination with QS21 and/or 3-D-MPL (or related molecules), enhances immune responses to a defined malaria antigen. Such enhancement available affords better immunological responses than hitherto before.

According to the present invention there is provided a vaccine composition comprising a Th1-inducing adjuvant in combination with a protecting Liver Stage Antigen or immunological fragment thereof of a human malaria parasite with the proviso that when the immunological fragment is an immunological fragment of LSA-3, the Thl-inducing adjuvant is not Montanide.

In a preferred aspect of the invention the Th1-inducing adjuvant comprises QS21, De-O-acylated monophosphoryl lipid A (3D-MPL) and an oil in water emulsion wherein the oil in water emulsion has the following composition: a metabolisible oil, such a squalene, alpha tocopherol and tween 80.

Normally the vaccine composition according to any aspect of the invention invokes a T cell response in a mammal to the antigen or antigenic composition and is preferably capable of stimulating interferon γ production. The oil in water emulsion used in the present invention may be utilised on its own or with other adjuvants or immuno-stimulants and therefore an important embodiment of the invention is an oil in water formulation comprising squalene or another metabolisable oil, alpha tocopherol, and tween 80. The oil in water emulsion may also contain span 85 and/or Lecithin.

The combination of the two adjuvants QS21 and 3D-MPL together with an oil in water emulsion is particularly preferred. This is known and referred to herein as SBAS2, or alternatively simply as AS2 or AS02.

The ratio of QS21: 3D-MPL will typically be in the order of 1:10 to 10:1; preferably 1:5 to 5:1 and often substantially 1:1. The preferred range for optimal synergy is 2.5:1 to 1:13D MPL: QS21. Typically for human administration QS21 and 3D MPL will be present in a vaccine in the range 1 μg-100 μg, preferably 10 μg-50 μg per dose. Typically the oil in water will comprise from 2 to 10% squalene, from 2 to 10% alpha tocopherol and from 0.3 to 3% tween 80. Preferably the ratio of squalene: alpha tocopherol is equal or less than 1 as this provides a more stable emulsion. Span 85 may also be present at a level of 1%. In some cases it may be advantageous that the vaccines of the present invention will further contain a stabiliser.

In an alternative preferred embodiment, the vaccine of the invention may advantageously comprise a vesicular adjuvant formulation comprising cholesterol, a saponin, and optionally an LPS derivative. In this regard the preferred adjuvant formulation comprises a unilamellar vesicle comprising cholesterol, having a lipid bilayer preferably comprising dioleoyl phosphatidylcholine, wherein the saponin and optionally the LPS derivative are associated with, or embedded within, the lipid bilayer. Preferably the vesicular adjuvant comprises both the saponin and the LPS derivative. More preferably, these adjuvant formulations comprise QS21 as the saponin, and 3D-MPL as the LPS derivative, wherein the ratio of QS21:cholesterol is from 1:1 to 1:100 weight/weight, and most preferably 1:5 weight/weight. Such adjuvant formulations are described in WO 96/33739 and EP 0 822 831 B, the disclosures of which are incorporated herein by reference. For example a suitable formulation may contain 0.25 mg cholesterol, 1 mg dioleoyl phosphotidylcholine, 5 ug 3D-MPL, and 50 ug QS21 and consist of small amellar vesicles wherein the saponin (QS21) and the LPS-derivative (3D-MPL) are in the membranes of the vesicles.

It will be appreciated that variants or derivatives of QS21 and 3-DMPL as described above may also be used without departing from the spirit of the invention.

The bacterial lipopolysaccharide derived adjuvants to be formulated in the adjuvant combinations of the present invention may be purified and processed from bacterial sources, or alternatively they may be synthetic. Accordingly, the LPS derivatives that may be used in the present invention are those inmmunostimulants that are similar in structure to that of LPS or MPL or 3D-MPL. In another aspect of the present invention the LPS derivatives may be an acylated monosaccharide, which is a sub-portion of MPL. In a preferred aspect the 3-DMPL is small particle 3-DMPL as described in WO 92/116556.

The oil emulsion adjuvants for use in the present invention may be natural or synthetic, and may be mineral or organic. Examples of mineral and organic oils will be readily apparent to the man skilled in the art based on the description hereinabove.

Particularly preferred oil emulsions are oil in water emulsions, and in particular squalene in water emulsions.

In addition, the most preferred oil emulsion adjuvants of the present invention comprise an antioxidant, which is preferably the oil o-tocopherol (vitamin E, EP 0 382 271 B1).

WO 95/17210 discloses emulsion adjuvants based on squalene, x-tocopherol, and TWEEN 80, optionally formulated with the immunostimulants QS21 and/or 3D-MPL.

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges. The amounts of the components present in the oil emulsions of the present invention are conventionally in the range of from 2 to 10% oil such as squalene; and when present, from 2 to 10% alpha tocopherol; and from 0.3 to 3% surfactant, such as polyoxyethylene sorbitan monooleate. Preferably the ratio of oil: alpha tocopherol is equal or less than 1 as this provides a more stable emulsion. Span 85 may also be present at a level of about 1%. In some cases it may be advantageous that the vaccines of the present invention will further contain a stabiliser. Preferably the oil emulsion contains a surfactant such as polyoxyethylene sorbitan monooleate (TWEEN80™), but it will be clear to the man skilled in the art that other surfactants may be used, preferred examples of which-are the SPAN series (especially SPAN85) and or lecithin.

The method of producing oil in water emulsions is well known to the man skilled in the art. Commonly, the method comprises the mixing the oil phase with a surfactant such as a PBS/TWEEN80™ solution, followed by homogerisation using a homogenizer, it would be clear to a man skilled in the art that a method comprising passing the mixture twice through a syringe needle would be suitable for homogenising small volumes of liquid. Equally, the emulsification process in microfluidiser (M110S microfluidics machine, maximum of 50 passes, for a period of 2 minutes at maximum pressure imput of 6 bar (output pressure of about 850 bar)) could be adapted by the man skilled in the art to produce smaller or larger volumes of emulsion. This adaptation could be achieved by routine experimentation comprising the measurement of the resultant emulsion until a preparation was achieved with oil droplets of the required diameter.

In a preferred aspect of the invention the human malaria parasite is Plasmodium falciparum.

In a particular aspect of the invention the said protecting Liver Stage Antigen is the Liver Stage Antigen 3 (LSA-3) or immunological fragment thereof.

However other Liver Stage Antigens may also be used, for example LSA-1 and LSA-2 as described in Malaria, Parasite Biology, Pathogenesis and Protection (1998 ASM Press, Washington D.C., edited by Irwin W. Sherman), especially Chapter 34 (P. Druilhe et al.).

By immunological fragment is meant herein a molecule which has a related or similar sequence to the reference antigen in terms of % homology and which can induce a similar immune response, cellular or humoral, in vivo.

The LSA-3 antigen and polypeptide molecules containing at least 10 consecutive amino acids of the amino acid sequence representing LSA-3 are described in WO 96/41877. LSA-3 for use in the present invention may suitably be prepared as described in the examples section of the present specification. Reference may also be made to C Marchand and P Druilhe, Bulletin of the World Health Organisation, Volume 68 (Suppl.) 158-164 (1990) and U.S. Pat. No. 6,100,067.

In a further aspect there is provided a vaccine composition according to the invention comprising in addition at least one other protecting antigen or an immunological fragment thereof, of a malaria parasite, in particular LSA-3.

In particular, the other malaria antigen may be selected from the following group:

a) a hybrid protein comprising substantially all the C-terminal portion of the CS protein, four or-more tandem repeats of the immunodominant region, and the surface antigen from hepatitis B virus (HBsAg), in particular RTS,S, or an immunogenic derivative including fragments thereof;

b) the TRAP protein of the T9/96 isolate of Plasmodium falciparum and proteins having at least 80% homology thereto and immunogenic derivatives including fragments thereof (see European Patent Application No 91903249.0);

c) the MSP-1 of Plasmodium falciparum or Plasmodium vivax and proteins having at least 80% homology thereto and immunogenic derivatives including fragments thereof; and

d) the MSP-3 of Plasmodium falciparum or Plasmodium vivax and proteins having at least 70% homology with the C-terminal region thereof, and immunogenic derivatives including fragments thereof.

MSP-1 of P.falciparum or P.vivax is described in U.S. Pat. No. 4,837,016. Immunogenic derivatives include fragments thereof such as the C-terminal 42 KDa antigen (p42).

The MSP-3 antigen is described in U.S. Pat. No. 6,017,538.

Homology in sequence analysis may be established by the use of Blast 2.0 and Fasta default settings of the algorithms used by these programs. The comparison of LSA-3 sequences in various isolates or stocks can be done using a calculation manual.

By C-terminal region of MSP-3 is meant a 185 amino acid region from positions 193 to 381. It contains a leucine zipper on its extremity (C-terminus part) and is rich in acidic amino acids. The three-dimensional structure is coil-coiled. The clone DG 210 (amino acids 193-257) corresponds to a globular region of high complexity and is followed by the coil-coiled region.

In a further aspect of the present invention there is provided a vaccine as herein described for use in medicine.

In yet a further aspect the invention provides a process for making a vaccine composition according to any aspect of the present invention by mixing the required components using standard techniques. Vaccine preparation is generally described in New Trends and Developments in Vaccines, edited by Voller et al., University Park Press, Baltimore, Md., U.S.A. 1978.

In one aspect the process comprises admixing QS21, 3D-MPL and the oil in water emulsion with a protecting Liver Stage Antigen of a human malaria parasite as hereinabove defined, optionally with an additional malaria antigen.

The amount of protein in each vaccine dose is selected as an amount which induces an inmmunoprotective response without significant, adverse side effects in typical vaccinees. Such amount will vary depending upon which specific immunogen is employed and how it is presented. Generally, it is expected that each dose will comprise 1-1000 ug of protein, preferably 2-100 ug, most preferably 4-40 ug. An optimal amount for a particular vaccine can be ascertained by standard studies involving observation of appropriate immune responses in subjects. Following an initial vaccination, subjects may receive one or several booster immunisation adequately spaced.

The formulations of the present invention maybe used for both prophylactic and therapeutic purposes.

Accordingly in one aspect, the invention provides a method of treatment comprising administering an effective amount of a vaccine of the present invention to a patient.

The following examples illustrate the invention.

### EXAMPLES

### Example 1

Two adjuvant formulations were made each comprising the following oil in water emulsion component.

SB26: 5% squalene 5% tocopherol 0.4% tween 80; the particle size was 500 nm size

SB62: 5% Squalene 5% tocopherol 2.0% tween 80; the particle size was 180 nm

1(a) Preparation of Emulsion SB62 (2 Fold Concentrate)

Tween 80 is dissolved in phosphate buffered saline (PBS) to give a 2% solution in the PBS. To provide 100 ml two fold concentrate emulsion 5 g of DL alpha tocopherol and 5111 of squalene are vortexed to mix thoroughly. 90 ml of PBS/Tween solution is added and mixed thoroughly. The resulting emulsion is then passed through a syringe and finally microfluidised by using an M110S microfluidics machine. The resulting oil droplets have a size of approximately 180 nm.

1(b) Preparation of Emulsion SB26

This emulsion was prepared in an analogous manner utilising 0.4,% tween 80.

To the emulsion of 1 a) or b) an appropriate amount of LSA-3 (for example 2 μg to 100 μg) may be added and mixed. This may be combined with, for example, 50 μg/ml of 3D-MPL and 20 μg/ml of QS21 (or related molecules) to give the final formulation.

### Example 2

Protection Against Plasmodiums Falciparum Malaria in Chimpanzees by Immunisation with a Conserved Pre-Erythrocytic Antigen, LSA-3

The basis of the strong immunological protection induced in humans by vaccination with radiation-attenuated pre-erythrocytic malaria parasites is poorly understood. However it is now suspected that the transformation of the irradiated sporozoites into live but developmentally arrested intra-hepatic liver trophozoites is required to induce protection⁹. This occurs at low (15-20 krad) but not at high (23-30 krad) irradiation doses^{9,10}. We reasoned that the differential response of hosts immunised with such irradiated sporozoites could provide a screen for molecules relevant to protection. We proceeded to screen 120 phage lambda clones previously identified as expressing P. falciparum polypeptides that are expressed during pre-erythrocytic stage parasite development and which derive from ca. 20 distinct genes^{6,7,11,12}. A clone corresponding to each of these putative genes was screened using eight sera from human volunteers (4/6 protected) and from chimpanzees (½ protected) immunised with sporozoites irradiated at low or high doses. A single clone (DG729) reacted only with sera from protected humans and chimpanzees. This differential reactivity was further confirmed with a peptide derived from this fragment (Table I). This led us to select this clone for further investigation.

DG129 was used to probe a P. falciparum (K1) genornic library. One clone was found to contain the whole gene corresponding to DG729, and which was named Liver Stage Antigen-3 (LSA-3). Full description of the sequence, expression, location and conservation of the lsa-3 gene is provided in the Supplementary Information (S.I.) and is summarised below and in FIGS. 1-3. Briefly we identified a single-copy gene which comprises a mini-exon 1, a mini-intron, and a large exon 2 (FIG. 1a), a structure similar to that of other surface antigens of P. falciparum¹³. It was recently confirmed that Lca-3 is located on chromosome 2¹⁴, where the gene was annotated as <<RESA-H3>> gene (Ace. Number AE001424). LSA-3, with a predicted molecular weight of 200 kDa (in K1), is made up of large non-repeated sequences flanking three glutamic acid-rich repeated regions, a feature that extends the known P. falciparum Glu-rich antigen network to include a pre-erythrocytic component The location of the original fragment (DG729) and of the peptides corresponding to the repeat region R2 and to the non-repetitive regions NR-A and NR-B are shown in FIG. 1b. Naturally- or artificially-induced antibodies against the non-repeated peptides and the recombinant protein GST-PC were not cross-reactive with the repeated Glu-rich regions, and were used for further studies.

Pre-erythrocytic expression of LSA-3 (see FIGS. 2-3 and see S.I.) was confutned a) by RT-PCR (primers i1 and i2) of total RNA and Western blotting of protein extracts, isolated in both cases from sporozoites, and b) by inimunofluorescence antibody test (IFAT) on infected liver sections and dry or wet sporozoite preparations, using antibodies to a non-crossreactive portion of the protein. In the five and six day-old liver schizonts, LSA-3 was located in the parasitophorous vacuole and at the periphery of maturing hepatic merozoites. This location is consistent with the molecular structure of this protein, which contains two hydrophobic regions (FIG. 1a). In our hands, mRNA from lsa-3 could not be detected in Northern blotted RNA from erythrocytic stages. Western blottings and IFAT of infected red blood cells were also consistently negative with non cross-reactive antibodies. Reactivity was however obtained when antibodies to the Glu-rich repeat region were used. This might explain in part the detection of a putatively homologous antigen (D260) previously described in intra-erythrocytic parasites, and which was identified solely using antibodies which cross-react extensively with Glu-rich epitopes¹⁶.

Polymorphism of many malaria vaccine candidate molecules is of recognised concern, we therefore investigated naturally occurring sequence variation in LSA-3 (see S.I.). The gene was consistently detected by PCR amplification of the NR-A region (primers S1 and S2) in a total of 111 P. falciparum isolates, strains or clones of various geographical origin. Using LSA-3 specific antibodies in IFAT assays, the expression of LSA-3 was also detected in liver schizonts of two distinct strains and in all the sporozoites from 30 wild isolates which developed in mosquitoes fed in vitro on Thai gametocytes. The repeat regions R1 and R3 are highly conserved, but variation in the number and order of the repeal units of R2 was found to occur amongst different parasite lines. This did not however affect the predicted conserved ?-helical organisation, a secondary structure considered to be important in defining major B-cell epitopes since antibodies which recognise R2 did indeed react positively by IFAT with all the parasites tested. The non-repeated portions of exon 2, where numerous Th and CTL epitopes are found¹⁷⁻¹⁹, displayed a remarkable degree of amino acid (an) sequence conservation between different parasites (>95.5% homology). The sequence of NR2 peptide was fully conserved amongst K1 and T9/96 parasites, the source of the immunising proteins, the NF54 parasites used for sporozoite challenges, and 27 P. falciparum samples of various geographical origin¹⁷. An HLA-B53 restricted epitope identified in the NR-B region of LSA-3 (present in GST-PC recombinant protein) was also found to be free of variation in clone 3D7 and in 18 —Gambian isolates¹⁹. This conservation of inmiunologically important epitopes contrasts with substantial polymorphism in current pre-erythrocytic vaccine candidates.

We selected the chimpanzee to investigate the protective capacity of LSA-3 immunisation for the following reasons. The chimpanzee is the only non-human primate fully susceptible to complete intra-hepatic development of P. falciparum, with a comparable rate of sporozoite transformation to liver forms to that seen in humans⁹. The chimpanzee is also the most closely related animal to humans (98.4% homology at the DNA level⁸), and one in which detailed investigations of immune responses can be performed and legitimately compared with those of humans¹⁷⁻¹⁸. The fact that parasitological and immunological events can be directly examined in the liver biopsies, a possibility excluded for infected humans, is clearly of considerable significance. A number of preliminary stringent tests were conducted in control animals in order to validate the suitability of this model for vaccine evaluation. Since cost and ethical considerations preclude the use of large number of animals, high reproducibility of the infection in this model system is critical. In a preliminary experiment (Group I, Table II), we confirmed that in the chimpanzee protection by inimnunisation with irradiated sporozoite is radiation dose-dependent, and we validated the detection of the infected red blood cells as an assay of protection. The results allowed us to define a number of important parameters: a) as in humans, chimpanzees develop a powerful protective response following immunisation with irradiated sporozoite, b) chimpanzees, like humans, remain broadly susceptible to at least five successive challenges, in contrast to lower primates or rodents which become refractory after the first challenge²⁰, and c) as a result of the high dose of inoculated sporozoites detection of erythrocytic parasites Corresponded to the first invasion of red cells by merozoites released from intra-hepatocytic schizonts. Positive blood smears were reproducibly obtained in non-protected chimpanzees on days six or seven, In the chimpanzee erythrocytic infections normally remain sub-clinical and self-limiting which was in fact observed despite the high dose challenges. These results have been recently confirmed in two further chimpanzees (Langermans J. et al, manuscript in preparation).

Having established the suitability of the chimpanzee, we proceeded to assay the protective value of LSA-3 immunisation by challenge with viable P. falciparum sporozoites. In preliminary experiments, two animals were inimunised with a mixture of LSA-3 and LSA-1 recombinant proteins. Full protection against three challenges over several months was only seen in the animal which responded to LSA-3 (both responded to LSA-1). In liver biopsies performed on this animal on day five, only one liver schizont of unhealthy appearance and infiltrated by leukocytes could be detected in the 300 liver sections screened (Dirk, FIG. 3). By contrast 2500 and 750 hepatic is schizonts of healthy appearance were observed in the two non-protected controls.

These results led us to focus further immunisation and challenge experiments on LSA-3 alone. Two groups of chimpanzees were used to evaluate lipopeptide and recombinant protein formulations (Table II, Groups II-III). In Group II, one animal (Gerda) was initially imxnunised solely with the NR2 lipopeptide of LSA-3, and boosted by recombinant LSA-3 molecules in Montanide ISA 51. Gerda was fully protected when challenged with 10⁷ sporozoites, whereas the control receiving Montanide ISA 51 was not (FIG. 4a).

In Gerda boosting with the recombinant LSA-3 formulation was not found to induce any detectable increase in the strong B-cell, T-helper cell and CTL responses already evoked by the initial lipopeptide/peptide injections^{17,18}. We were therefore interested to see whether the simple and well-tolerated peptidic formulation alone could induce protection. Two chimpanzees, Mopia and Mgbado were imniunised with LSA-3 lipopeptides/peptides alone (Table II, Group III). Protection against a first challenge with 2×10⁴ sporozoites was obtained in both. The same group included an investigation of the effects of microbead presentation of recombinant proteins without adjuvant in one animal (Judy) which resulted in a one-day delay to patency (FIG. 41,). Following a subsequent high dose sporozoite challenge (5×10⁶ sporozoites), both Mopia and Mgbado demonstrated a clear two-day delay to patency and a low transient parasitaemia, whilst no protection was found for Judy (FIG. 4c). The delay to patency suggests that the immune responses had caused a reduction exceeding 90% of intra-hepatocytic schizont load²¹ (FIG. 4).

In chimpanzees from groups IV and V, we investigated the efficacy of a less complex lipopeptide mixture alone, or of recombinants adjuvated by SBAS2, a novel adjuvant whose efficacy has been recently established in humans^{4,5}. Since immunogenicity studies^{17,18} and analysis of previous chimpanzee data had indicated that peptide CII was poorly immunogenic and thus might not be critical, chimpanzee Patty was immunised by a mix of three instead of four peptides. This animal showed protection upon challenge. Among four animals receiving SBAS2 adjuvated LSA-3 proteins, two showed full, sterile protection against a medium dose challenge. One showed a delay in patency which may be indicative of partial protection, whereas neither the fourth nor the control receiving SBAS2 adjuvant alone were protected. One of the two fully protected chimpanzees was further challenged with a high dose three months later and still showed full protection.

We present here the first description of protective vaccination against human malaria in the chimpanzee. This model provided us with convincing evidence that LSA-3 of P. falciparurn is a valuable candidate for effective vaccination against pre-erythrocytic stages. A total of nine animals were immunised using lipopeptides in saline or polypeptides in either Montanide or SBAS2 adjuvants. Full sterile protection was induced in six of these nine chimpanzees on first challenge. If the significant delay as compared to controls is taken in consideration, a protective effect induced by LSA-3 was shown in eight of nine animals. Out of the 14 challenges which were performed, complete protection was obtained in seven, and partial protection in an additional four challenges. All seven control animals employed in these studies showed a-consistent pattern in the appearance and the course of the blood-stage parasitaemiae following each of the 12 challenges with viable parasites. Demonstration of this reproducibility in controls, in animals immunised by over-irradiated sporozoites, and in an additional 26 challenges performed in other experiments (not shown), is an essential point in the interpretation of our data.

It is encouraging that protection was induced against a heterologous challenge (NF54) in outbred animals immunised with LSA-3 molecules whose sequences were derived from K1 and T9/96 parasites. A variety of imnmunisation strategies were investigated in the course of this work. The data underpin the value of the SBAS2 adjuvant. The results with Gerda, Mopia, Mgbado and Patty are also particularly encouraging since they are based on simple peptide and lipopeptide formulations which are relatively easy to produce under GMP conditions²². In our animals no local or general reactions was detected following lipopeptide injections, an observation consistent with previous experience with similar formulations derived from SW in macaques²³ and HbS²⁴ or HIV²² in humans. This bodes well for future clinical trials.

Methods

Selection of clone DG729. Dot blot analysis of the β-galactosidase-fused-recornbinant proteins encoded by the pre-erythrocytic clones was performed on nitrocellulose as previously described⁷, using 1/100 diluted human and chimpanzee scm. ELISA was performed in duplicate as previously described²⁵ on 1,100 diluted sera using coating solutions of 0.3, 3 and 10 μg/ml of NR1, NR2 and RE peptides respectively, in PBS.

LSA-3 cloning and characterisation. Detailed description of molecular methods, gene cloning, sequence data, protein characteristics and description of the recombinant proteins and of the peptides are provided in the S.I. The primers used for PCR: S1 (nucl.161-184)/S2 (nucl.454-432) and for RT-PCR: i1 (nucl.695-722)/i2 (nucl.824-799), numbering refers to the lsa-3 sequence of K1 (Accession Nber AJ007010). All mouse sera used for the Western blot (at dilution 1/100) presented in FIG. 2 were obtained following 3 subcutaneous injections of the ininiunogen (100 μg) emulsified in SBAS2 adjuvant⁴. Long synthetic peptides GP5, GP6, GP8 and GP11 were synthesised as described in ref. 26 (see FIG. 1 for position).

Immunogens injected in chimpanzees. Sequences of the various immunogeris evaluated here consisted of clone DG729 and inserts NN and PC, as well as peptides (pep.) NR1, NR2, RE and CT1; their location is shown in FIG. 1 and described in more details in the S.I. Clone DG729, as well as inserts NN and PC were expressed as glutathione-S-transferase-fused recombinants and purified according to manufacturer recommendations (Invitrogen, The Netherlands). Recombinants GST-DG729,-NN and -PC were designed so as to cover 95% of the LSA-3 antigen and were used as a mixture mentioned as LSA-3 GST-rec. Peptides NR1, NR2 and CT1, were also synthesised as pahnitoyl-conjugated lipopeptides (lipopep.), as described in ref. 17. Combination of synthetic compounds (mentioned as (lipo)pep.) consisted in a mixture of NR1, NR2 and CT1 lipopeptides and of RE peptide. All peptides and lipopeptides were purified to >90% purity by reversed-phase chromatography, and the impurities consisted essentially of related peptides of shorter sequences¹⁷.

Chimpanzee immunisations and challenges. None of the chimpanzees included in this study hail previously been exposed to malaria infections or malarial antigens. Recombinant and synthetic compounds were-injected subcutaneously, at a dose of 100 μg for each peptide and/or lipopeptides, and/or 50 μg for each protein. Lipopeptides were always injected in PBS and, except when mentioned, peptides and recombinants were emulsified in Montanide ISA51. Group I animals (Carl and Japie) were immunised by five intra-venous injections of 5×10⁶ gamma-irradiated sporozoites at day 0 and weeks 8, 24, 44 and 65, and received three challenges at weeks 71, 97 and 123 (challenge doses are given in Table II). One year after the three challenges reported here, these chimpanzees were re-immunised once, and received one low and one high dose challenges, which revealed the same pattern of protection (not shown, Langermans J. et al., manuscript in preparation). In Group II, Gerda received NR2 lipopeptide at day 0 and weeks 3, 13 and 31 as described in ref. 17. She was then boosted with the mixture of LSA-3 GST-rec. at weeks 40,45,48 and 50. Control animal Lianne received Montanide ISA51. Challenges were performed at week 60. Group III animals were immunised at day 0 and weeks 3 and 6. Mopia and Mgbado received LSA-3 (lipo)peptides whereas Judy was injected with LSA-3 (3ST-rec. adsorbed to latex miorobeads. Challenges LD and HD were performed at weeks 21 and 29. In Group IV, Patty received LSA-3 (lipo)peptides, but without lipopeptide CT1, whereas Wendy and Willy were injected with LSA-3 GST-rec in SBAS2 adjuvant^{4,5}. Control animal Helen received SBAS2 adjuvant only. All animals were immunized at weeks 0, 4 and 8 and were challenged with 20,000 sporozoites at week 13. In Group V, Cindy and Marty were both immunised at weeks 0, 4, 8 and 26 with TSA-3 GST-rec in SBAS2 adjuvant (as in Group IV) and negative control animal Fauzi received over-irradiated sporozoites similarly to Sapie (Group I) at weeks 5, 8, 11 and 26. Challenges LD and HD were performed at weeks 33 and 46 in all three animals.

NF54 sporozoites were obtained from dissected salivary glands of infected Anopheles gambiae as previously described²⁷. Sporozoites were pooled, resuspended in PBS and injected intravenously. All animals in each group were challenged with the sante pool of sporozoites. For cost reasons, extensive evaluation of the Minimal Infective Dose has not been undertaken, however challenge with 5×10³ sporozoites, the lowest dose used to date, has proven infective in four other animals (Thomas, A. W., unpublished data).

Determination of the protective status. For Groups I, II, IV and V, animals blood was taken on days five to nine, and evaluated by thick and thin film Gienisa-stained preparations, and confirmed in all cases by in vitro culture (not shown), as described in ref. 21. For Group III chimpanzees blood taken every day from day five up to day 18, then every other day up to day 30, was used to prepare thin and thick smears which were Giemsa-stained and examined by two separate microscopists. A chimpanzee was considered a) totally protected when no parasites could be detected in the circulation blood, by direct microscopical observation and by long term culture, or b) partially protected when time to patency was delayed by one or more days as compared to that observed in control animals. In mice, these delays correspond to a protection of 80% (24 h) or 96% (48 h) against sporozoite challenges. In humans, a 12 hour delay was calculated to correspond to a 92% reduction of liver forms following sporozoite challenges²¹. In a limited number of animals a liver biopsy was performed under anaesthesia by a veterinary doctor on day five following a high dose challenge. Material was fixed and 4 μm sections were made and stained by Gietnsa-collophonium²⁸ before complete microscopic enumeration of the liver forms in 300 sections (average area 0.8 cm²). All animals were curatively treated with chloroquine immediately after the period of observation, and irrespective of their protective status.

### REFERENCES TO EXAMPLE 2

1. Herrington, D., el al. Successful immunization of humans with irradiated malaria sporozoites: humoral and cellular responses of the protected vaccinees. Am. J. Trop. Med. Hyg. 45, 539-547 (1991).

2. Egan, I. E., et al. Humoral immune responses in volunteers immunized with irradiated Plasmodium falciparum sporozoites. Am. J. Trop. Med Hyg. 49, 166-73 (1993).

3. Facer, C. A. & M., Tanner. Clinical trials of malaria vaccines: progress and prospects. Adv. Parasitol. 39, 1-68 (1997).

4. Stoute, J. A., et al. A preliminary evaluation of a recombinant circumsporozoite protein vaccine against Plasmodium falciparum malaria. New Engl. J. Med. 336, 86-91(1997).

5. Stoute, J. A., et al. Long-term efficacy and immune responses following immunization with the RTS,S malaria vaccine. J. Infect Dis. 178, 113944 (1998).

6. Guérin-Marchand, C., et al. A liver stage-specific antigen of Plasmodum falciparum characterized by gene cloning. Nature. 329, 164-167 (1987).

7. Marchand, C. & Druilhe, P. How to select Plasmodium falciparum pre-erythrocytic antigens in an expression library without defined probe. Bull. WHO. 68 (suppl.), 158-164 (1990).

8. Miyaxnoto, M. M., Koop, B. F., Slightom, I. L., Goodman, M. and M. R., Tennant. Molecular systematics of higher primates: genealogical relations and classification. Proc. Nat. Acad Sci. U.S.A. 85, 7627-31 (1988).

9. Druilhe, P., et al. in “Malaria. Parasite Biology, Pathogenesis and Protection” (eds. Irwin W. Sherman), p.513-543 (American Society for Microbiology, Washington D.C., 1998).

10. Mellouk, S., Lunel, F., Sedegah, M., Beaudoin, R. L. and P., Druilhe. Protection against malaria induced by irradiated sporozoites. Lancet. 335, 721 (1990). the circuntsporozoite protein retards infection. J. Gun. Microbial. 27, 1434-1437 (1989).

22. Gahery-Segard, H., et cd. Multiepitopic B- and I-cell responses induced in humans by a Human Immunodeflciency Virus type 1 lipopeptide vaccine. J. Viral. 4, 1694-703 (2000).

23. Bourgault, I., et al. Simian immunodeficiency virus as a model for vaccination against HIV: induction in rhesus macaques of GAG or NEF specific cytotoxic T lymphocytes by lipopeptides. J. Immunol. 152, 2530-2537 (1994).

24. Vitiello, A., et al. Development of a lipopeptide-based therapeutic vaccine to treat chronic HBV infection. Induction of a primary cytotoxic T lymphocyte response in humans. J. Clin. Invest. 95, 341-349 (1995).

25. Londoño, J. A., Gras-Masse, H., Dubeaux, C., Tartar, A. and P., Druilhe. Secondary structure and immunogenicity of hybrid synthetic peptides derived from two Plasmodium falciparum pre-erythrocytic antigens. J. Immunol. 145, 1557-1563 (1990).

26. Roggero, M. A., et al. Synthesis and immunological characterization of 104-mer and 102-mer peptides corresponding to the N- and C-terminal regions of the Plasmodium falciparum CS Protein. Mol. Jmmunol. 32, 1301-1309 (1995).

22. Ponnudurai, T., at al. Sporozoite load of mosquitoes infected with Plasmodium falciparum. Trans. Roy Soc. Trap. Med Hyg. 83, 67-70 (1989).

28. Druilhe, P., Puebla, R. M., Miltgen, P., Perrin, L. and M., Gentilini. Species- and stage-specific antigens in exoerythrocytic stages of Plasmodium falciparum. Am. J. Trap. Med Hyg. 33, 336-341 (1984).

29. Meis, J. F. G. M., et al. Plasmodium falciparum: studies on mature exoerytbrocytic forms in the liver of the chimpanzee, Pan troglodytes. Exp. Parasitol. 79,1-11(1990).

### Example 3

The following experiments take advantage of the long peptide strategy (LSP) developed by GianPietro Conadin in Lausanne, which allow one to establish proof of

11. Fidock, D. A., et al. Cloning and characterization of a Plasmodium falciparum sporozoite surface antigen—STARP. Mol. Biochem. Parasitol. 64, 219-232 (1994).

12. Bottius, E. et al. A novel Plasmodium falciparum sporozoite and liver stage antigen (SALSA) defines major B, I helper, and CTL epitopes. J. Immunol. 156, 2874-2884 (1996).

13. Kemp, D. J., Cowman, A. F. and D., Walliker. Genetic diversity in Plasmodium falciparum. Adv. Parasitol. 29, 75-149 (1990).

14. Gardner, M. J., at al. Chromosome 2 sequence of the human malaria parasite Plasmodium falciparum. Science, 282, 1126-1132 (1998).

15. Moelans, I. I. M. D. & J. G. G., Schoenmakers. Crossreactive antigens between life cycle stages of Plasmodium falciparum. Parasitol. Today. 8, 118-123(1992).

16. Barnes, D. A., Wollish, W., Nelson, R. G., Leech, J. H. and C., Petersen. Plasmodium falciparum: D260, an intraerythrocytic parasite protein, is a member of the glutamic acid dipeptide-repeat family of proteins. Exp. Parasitol., 81, 79-89 (1995):

17. Ben Mohamed, L., et al. Lipopeptide immunization without adjuvant induces potent and long-lasting B, I helper, and cytotoxic T lymphocyte responses against a malaria liver stage antigen in mice and chimpanzees. Eur. J. Immunol. 27, 1242-1253 (1997).

18. Ben Moharned, L. et al. High immunogenicity in chimpanzees of peptides and lipopeptides derived from four new Plasmodium falciparum pre-erythrocytic molecules. Vaccine, 18, 2843-2855 (2000).

19. Aidoo, M., et al. CTL epitopes for HLA-B53 and other HLA types in the malaria vaccine candidate Liver Stage Antigen-3. Infect. Immun. 68, 227-232 (2000).

20.Nüssler, A. K., et al. In viva induction of the nitric oxide pathway in hepatocytes after injection with irradiated malaria sporozoites, malaria blood parasites or adjuvants. Eur. J. Immunol. 23, 882-887 (1993).

21. Murphy, J. R., Baqar, S., Davis, J. R., Herrington, D. A. and D. F., Clyde. Evidence for a 6.5-day minimum exoerythrocytic cycle for Plasmodium falciparum in humans and confirmation that immunization with a synthetic peptide representative of a region of concept at clinical level by producing in short time and at low cost Long Synthetic Peptides. These are in fact short proteins which can be employed in clinical trials. A series of 17 overlapping Long Synthetic Peptides was synthesised covering the full length of the LSA-3 molecule.

These peptides were used in antigenicity studies at T-cell and B-cell level in exposed individuals in the field in Senegal to monitor antibody and lymphoproliferative responses to each of them in exposed populations. They were used also to immunise mice using AS2 adjuvant.

Both studies demonstrated a very strong antigenicity of most of the peptides which, each, defined at least one B-cell and one T-cell epitope and immunogenicity studies in mice indicated that most peptides studied were also strongly immunogenic to laboratory mice (summarised in: Perlaza at al. European Journal of Immunology, 2001 Jul;31,7,2200-9)

Challenge experiments with the cross-reactive Plasmodium of rodents, Plasmodium yoelii, indicated in particular that a peptide called GP1 could induce protection against virulent P.yoelii sporozoite challenge. For further studies in humans, to chose the irnmunizingg peptides we relied on initial results obtained with the recombinant denominated DG729 which overlaps part of the non-repetitive N-terminal region of the molecule and the beginning of the repeat region.

Two types of formulations were investigated:

a) A very long LSP of ca 160 aminoaeids, covering the end of the Non-repeated region, including the short peptides NR1 and NR2 investigated formerly and the beginning of the repeat region.

b) A mix of 2 peptides, one covering only the non-repeat region, called GP1 and another, called GP14, located in the beginning of the repeat region. For practical reasons, it was found that it would be difficult to produce in sufficiently pure form the very long species mentioned above in a), and that for GMP production it would be safer to rely on a mix of the two peptides mentioned in b), namely GP1 and GP14.

Therefore, pre-clinical studies were performed in South-american primates, Aotus trivirgatus griseimembra, by Blanca-Liliana Perlaza in the collaborative laboratory of Socrates Herrera in Cali, Colombia.

7 Animals were Enrolled in this Study as Follows:

Group 1: 2 animals receiving 3 injections of LSA-3 GPI LSP at a dose of 50 mg pet injection per animal, adjuvated by AS2 in a total volume of 500 ml per injection.

Group 2: 2 animals receiving 3 injections of a mixture of peptides LSA-3 GP1 SA-3 GP14, at a dose of 50 mg of each peptide per injection per animal, adjuvated by AS2 in a volume of 500 ml per injection.

Group 3: 2 animals receiving 3 injections of PBS with adjuvant AS2 in a volume of 500 ml per injection, plus ] non-immunised control.

One month after the-last immunisation, which were well tolerated and did not induce any major local or general reactions, blood samples were taken to analyse immunogenicity: results are shown in the corresponding graphs and demonstrated strong antibody production, lymphoproliferative responses and Interferon-g production, both in culture supernatant of lymphocytes and by Elispot technique. Animals were challenged by intra-venous inoculation of 100 000 sporozoites of the Santa Lucia strain of Plasmodium falciparium 3 months after the last immunisation Blood samples taken over a period of 60 days after challenge may be analysed by 3 different techniques, namely microscopy of coloured blood, Polymerase Chain Reaction and LDH assay.

The study of the degree of protection achieved by the LDH assay has been completed. This method relies on the detection of the parasite by a double-site ELISA capture assay which has been recently described (Druilhe et al., American Journal, 64 (5, 6) 2001, 233-241) and which was shown to be at least 10 times more sensitive than microscopy. The results obtained are presented in the figures. They essentially show that the 3 control animals became blood-stage positive, i.e. yielded positive parasite-specific LDH detection during the follow-up, whereas the 2 immunised groups remained consistently negative by this technique over the 60 days of follow-up.

The results support the protection induced by immunisation by the GP1 LSP or the GP1+GP14 LSPs adjuvanted by AS2. These results are in agreement with previous data obtained using the recombinant DG729 alone which covers the same region of the antigen, as well as immunisation performed by a mix of lipopeptides coveting the same region, as well as those obtained by a mix of 3 recombinants (729, NN, PC) adjuvanted by AS2 (Daubersjes et al., Nature Medicine, Nov. 2000, 6, 11, 1258-1263). The sequence of the 2 peptides employed is shown below.

GP1
L A S E E V K E K I L D L L E E G N T L T E S V D
D N K N L E E A E D I K E N I L L S N I E E P K E
N I I D N L L N N I G Q N S E K Q E S V S E N V Q
V S D E L F N E L L N S V D V N G E V K E N I L E
E S Q V N D D I F N S L V K S V Q Q E Q Q H N
GP 14
ESVAENVEES VAENVEEIVAPTVEEIVAPTVEEIVAPSVV
ESVAPSVEES VEENVEESVA ENVEESVAEN
VEESVAENVEESVAENVEEI VAPTVE

| Code | Spz. | IFAT | | NR2 |
|---|---|---|---|---|
| or | irrad. | titers | | peptide |
| Name | dose | on spz. | status | (aa 198-223) |
| V4 | 23.6 | 4,096 | not protected | 0.5 |
| V5 | 23.6 | 32,000 | 2 day delay | 0.5 |
| Japie | 30 | 3,200 | not protected | 0.7 |
| V6 | 20.8 | 5,120 | Protected | 3.8 |
| V7 | 20.8 | 41,960 | Protected | 2.6 |
| V8 | 20.8 | 40,960 | Protected | 4.8 |
| WR4 | 15 | 3,200 | Protected | 3.4 |
| Carl | 18 | 6,400 | Protected | 2.3 |

Spz.: sporozoite;
irrad.: irradiation

Table I (above): Differential reactivity of sera from protected or non-protected humans or chimpanzees with peptide NR2. IgG-specific antibodies against peptide NR2 were measured by ELISA in sera from human volunteers (codes) and chimpanzees (names in italic) immunised with sporozoites irradiated at low or high dose (in krad). Codes, immunisation schemes, sporozoite IFAT titres and protective status determination for human volunteers V4-V8 and WR4 are detailed in ref. 1 and 2, respectively. Chimpanzees Carl and Japie were inirnunised and challenged as described in the text and the Methods (Group I). ELJSA titres are expressed in arbitrary units representing the ratio of the mean ODs from test sera to the mean OD plus three standard deviations from 10 controls studied in parallel iii the same plate. Results are taken as positive for ratios above one (in bold). Similar experiments performed with peptides NR1 and RE (see FIG. 1) yielded negative results with these sera (not shown).

| | | | PROTECTION | |
|---|---|---|---|---|
| ANIMAL GROUPS | | Immunisation and challenge | LD | HD |
| Chimp. | Immunisation protocols^{a} | dates (weeks) | 2 × 10⁴ | 10⁷ |
| Carl Japie Marcel Theo | Group I^{b 18 krad-irradiated sporozoites 30 krad-irradiated sporozoites unimmunised control unimmunised control} | 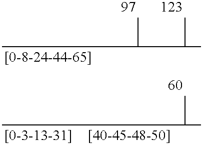 | +−−− | +−−− |
| Gerda Lianne | Group II [lipopep. NR2] then [GST-rec. in ISA51]control ISA 51 | 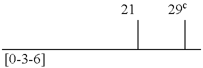 | nd nd | +− |
| Mopia Mgbado Judy Ondele Makata | Group III [(lipo)pep.][(lipo)pep.][GST-rec./microbeads]control GST/microbeads unimmunised control | 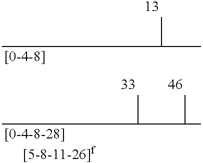 | ++d1 −− | d2 d2 −−− |
| | Group IV | | | |
| Patty | [(lipo)pep.]^{d} | | + | nd |
| Wendy | [GST-rec. in SBAS2] | | + | nd |
| Willy | [GST-rec. in SBAS2] | | − | nd |
| Helen | control SBAS2 | | − | nd |
| | Group V | | | |
| Cindy | [GST-rec. in SBAS2] | | + | + |
| Marty | [GST-rec. in SBAS2] | | d1 | − |
| Fauzi | 30 krad-irradiated sporozoites | | − | − |

Chimp.: chimpanzee name; HD/LD: high/low dose sporozoite challenges; d1/d2: one/two-day delay to patency; nd: not done.
^{a}details and abbreviations are given in the Methods.
^{b}Group I chimpanzees received three additional challenges (2 LD and 1 HD) which led each time to similar results, i.e. a reproducible protection only in Carl (data not shown).
^{c}HD challenge was performed with 5 × 10^{6 sporozoites.}
^{d}same mixture as in Group III but without peptide CT1.
^{e}performed in Cindy and Marty.
^{f}performed in Fauzi.

Table II (above): Immunisation and challenge experiments in the chimpanzees. Challenges were performed with either 2×10⁴ (low dose) or 10⁷ (high dose) NF54 P. falciparium sporozoites (“Protection” column). Immunisation schedules (in brackets under the bar) and of challenges (indicated by arrows above the bar) are expressed in weeks from first immunisation. Complete protection is indicated with (α); a delay to patency (in days) as compared to controls and non-protected animals is indicated by d1 or d2 (determination of the protective status is detailed in the Methods).

### LEGENDS FOR FIGURES

FIG. 1: Schematic representation of the LSA-3 gene, recombinant proteins and peptides. a) 6.2 Kb Eco RI-insert isolated from K1 parasite genomic DNA library that hybridised with DG729. The 5.53 Kb gene comprises a 198 bp exon 1, a 168 bp intron (i) and a 5.16 Kb exon 2. Regions NR-A, -B and -C correspond to non-repeated sequences whereas R1 to R3 designate the three repeat blocks. The two hydrophobic regions potentially corresponding to the NH2-terminal signal peptide and the anchor region are indicated by HR1 and HR2 respectively. b) Location of the sequences encoding for LSA-3 in the recombinant fusion proteins (first line) and the synthetic peptides (strokes) used in this study (see Supplementary Information for aa numbering). For the immunisations, CT1 and NR2 were also used as palmltoyl-conjugated lipopeptides¹⁷ (indicated by *).

FIG. 2: LSA-3 expression in P. falciparium sporozoites. Western blot analysis was performed on protein extracts from NF54 sporozoites and control uninfected mosquito salivary glands using mouse antisera directed against: C) control GST, I) GST-PC, 2) peptides GP5, GP6, GP8 or GP11, 3) GST-729 (see FIG. 1, Methods and S.I.). LSA-3 is visualised as a 175 kDa protein (*), in agreement with the theoretical molecular weight of LSA-3 in this parasite strain.

FIG. 3: Immunostaining of P. falciparium pre-erythrocytic stages with anti-LSA-3 antibodies. a) sporozoites stained by IFAT with human antibodies affinity purified on recombinant βgal-DG729. b) staining by IFAT of day six post-challenge liver stages from a chimpanzee, using the antibodies induced by lipopeptide NR2 injection¹⁷ in chimpanzee Gerda (see S.I. for additional pictures). c) The single residual liver schizont detected in a chimpanzee Dirk (day five post-challenge) appeared infiltrated by lymphomononuclear cells, as compared in d) to one of the numerous healthy schizonts observed in the control chimpanzee Peer (total of ca 2500 scbizonts/300 liver sections. Giemsa-collophonium staining²⁸) (see text). Bars correspond to 5 μm in panel a) and 20 μm in panels b) to d).

FIG. 4: Blood parasitaemia courses in Groups II and III. a) chimpanzees from Group II and b-c) animals in Group III, following high dose (HID) or low dose (LD) challenges with NF54 sporozoites. Names of totally or partially protected animals are in bold. Hatched patterns correspond to control chimpanzees. Parasitaemia scales are different for each challenge, as expected from challenges with different numbers of sporozoites. Note that the day of patency in control and non-protected animals was the same for a given challenge inoculum within each group (in the above and in other groups not shown here).

FIG. 5: antibody titers following immunisation with GP1 and GP14

The figure shows the results from ELISA assays of Aotus M73 and D114 immunised by LSA-3 GP1+adjuvant AS2 against the immunising peptide GP1 or the recombinant DG729. In both cases, the titers are high as the result is significant for values higher than an ELISA ratio of 1. The second half of the figures show the results obtained in Aotus M88 and M91 inimunised by GP1+GP14 adjuvated by AS2, against peptide GP1 and GP14 or the recombinant 729 or NN covering the repeat region, or a control recombinant (GST). Here again, the responses are high against both immunising peptides.

FIG. 6: Proliferative Responses to GP1 and GP14

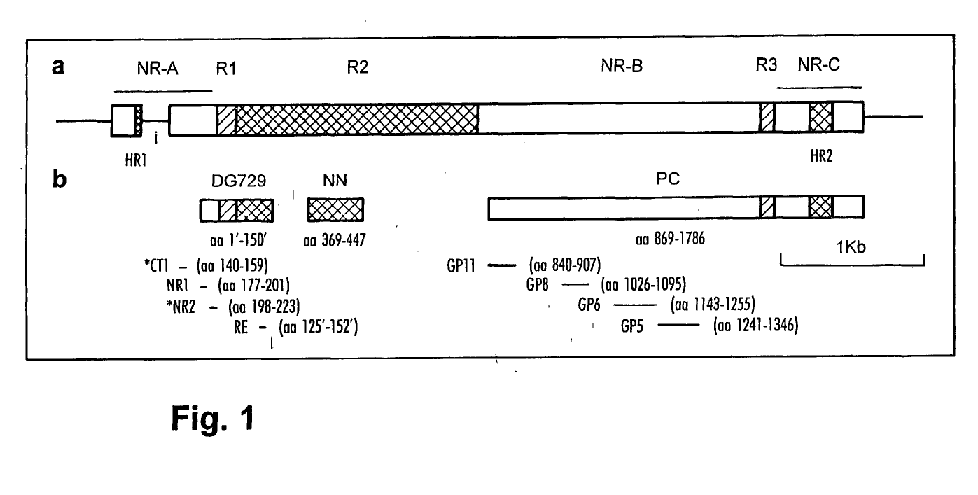

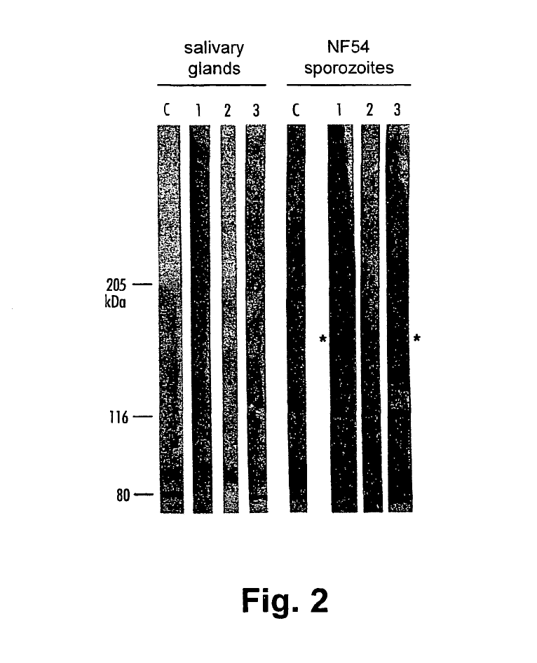

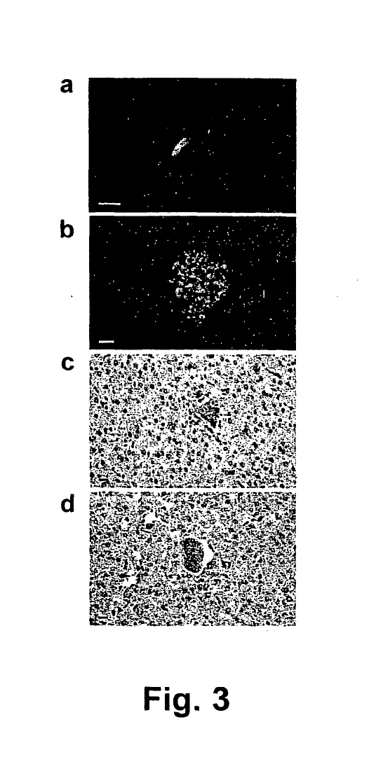

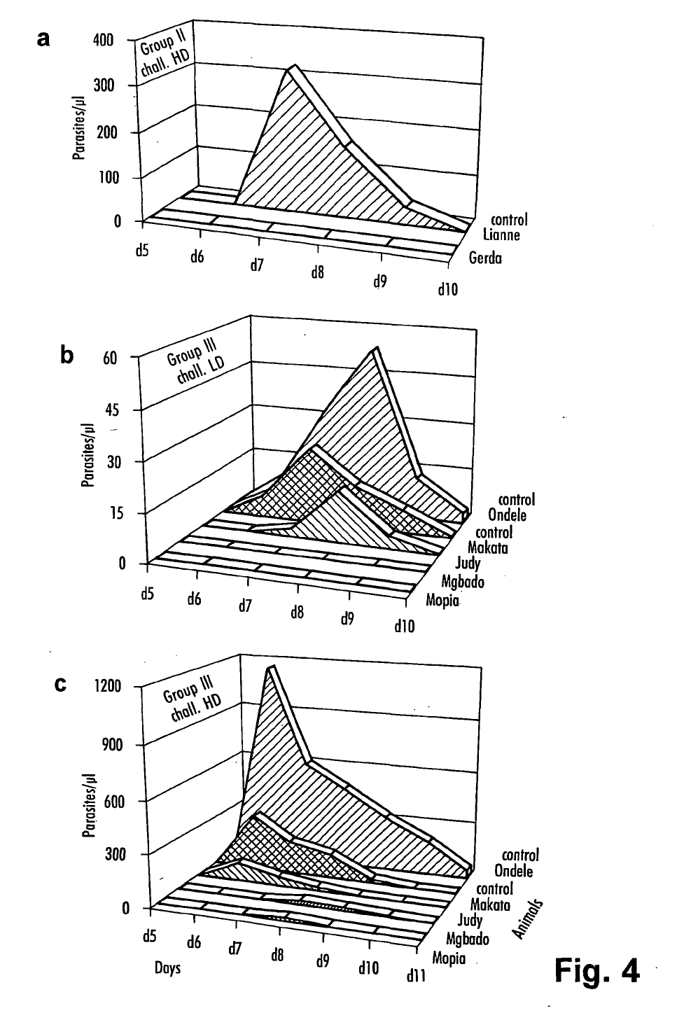

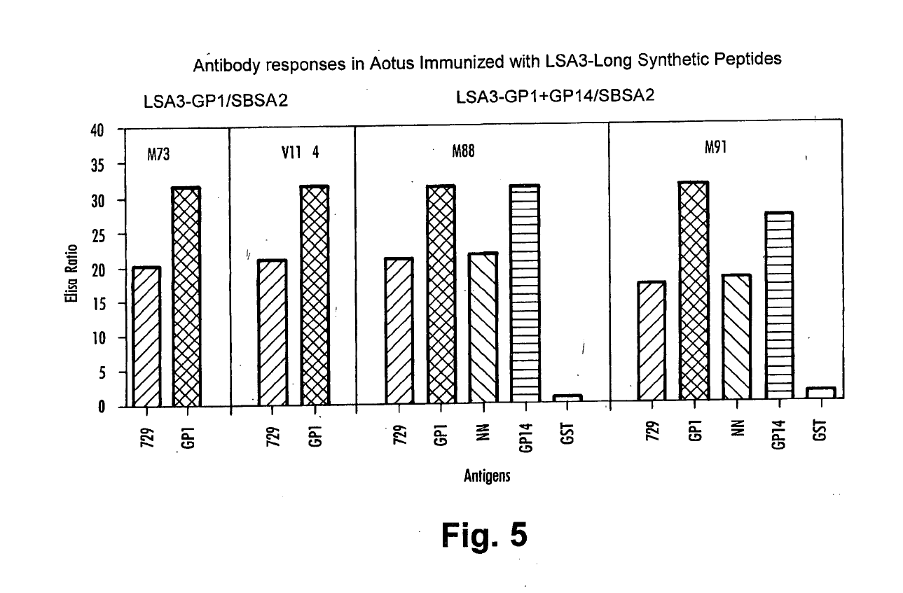

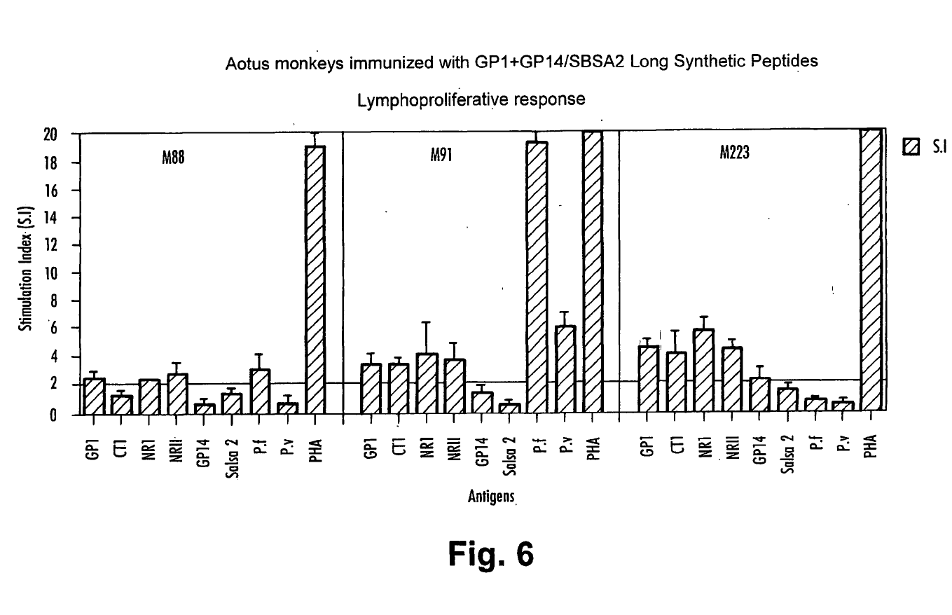

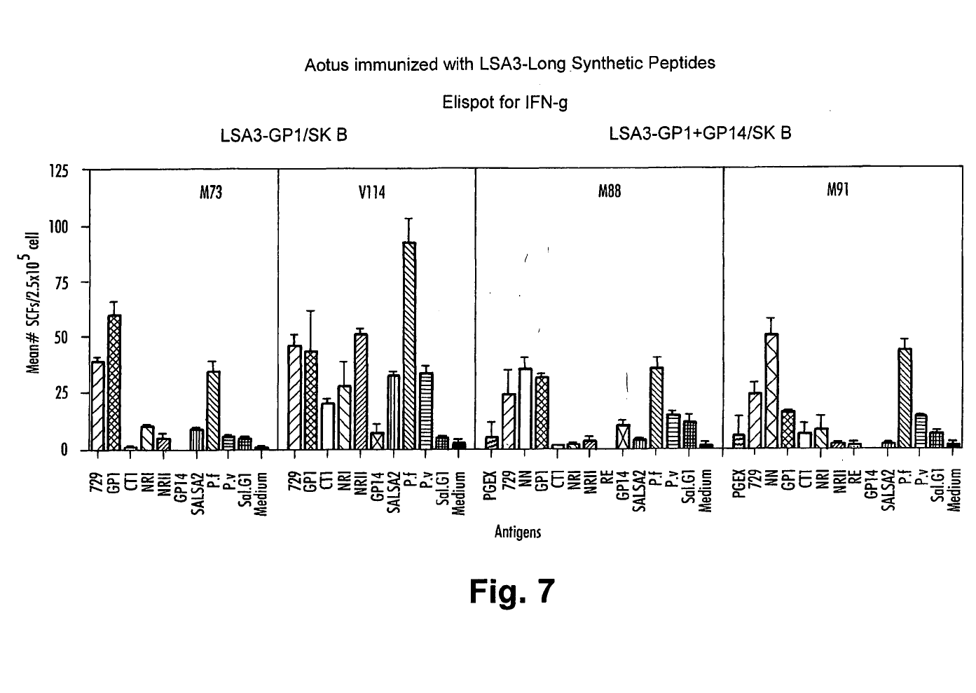

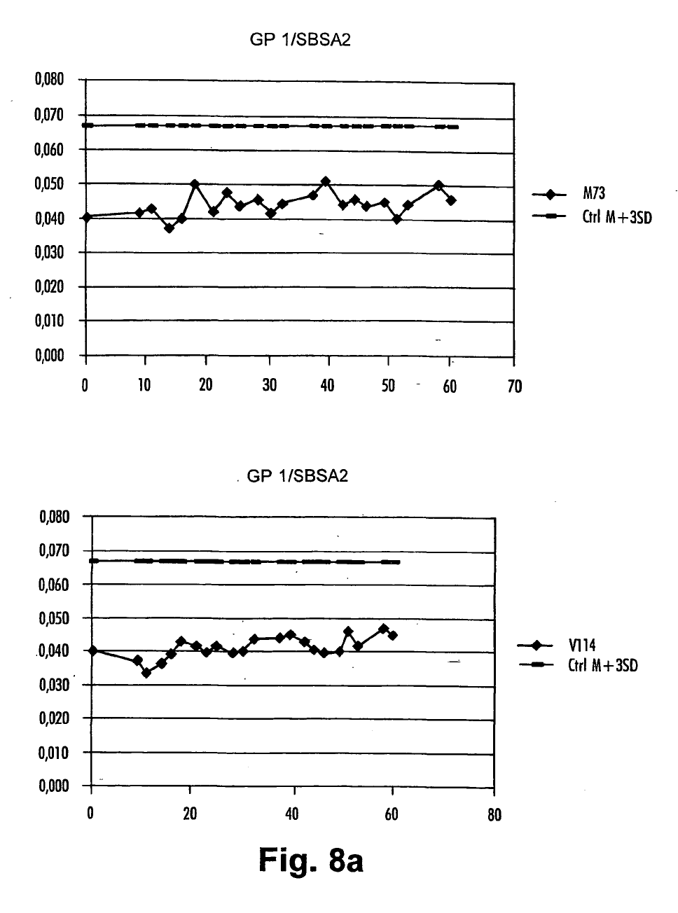

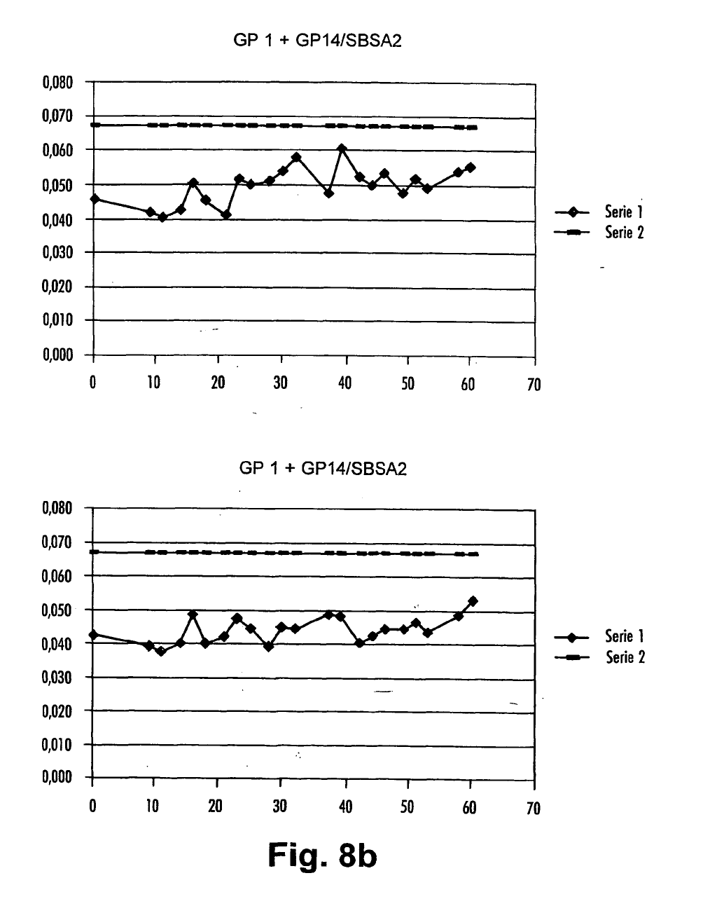

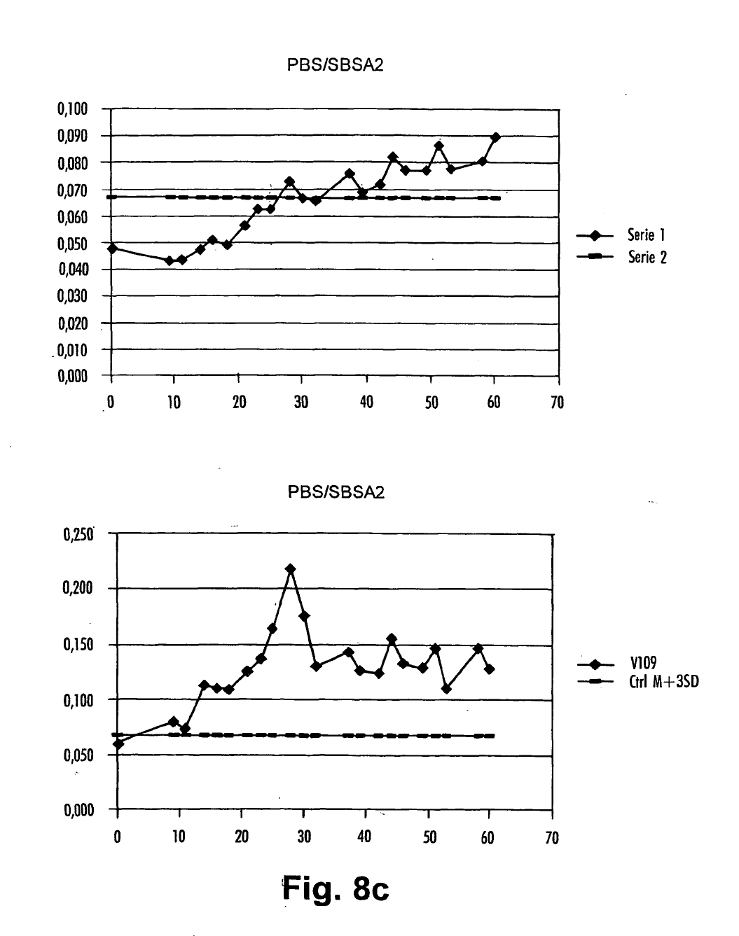

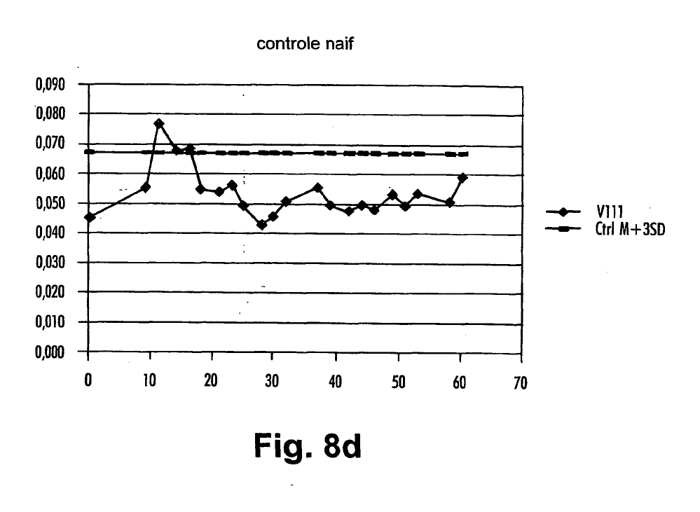

The figure shows the proliferative responses in M88 and M91 and M223 (a third animal, included in fact in group 2, but not challenged) of monkeys immunised by a mix of GP1+GP14 with AS2 adjuvant. Significant proliferative responses were obtained to the immunising peptide GP1 and, to a lesser extent, GP14, or to smaller peptides here contained in the longer ones such as NR1 NR2, or the sporozoites themselves (Pf). However, responses were lower and borderline (threshold of positivity=2) in monkey M88 as compared with the 2 others. The PHA is a positive stimulation control.

In monkeys immunised only by GP1 adjuvanted by AS2, positive responses were mainly recorded in monkey M73 and were only borderline positive to the immunising peptide GP1 in monkey V114 (whereas they are essentially negative in monkey M51).

FIG. 7: Elispot Assays

The figure shows responses recorded in monkey M73 and V114 receiving GP1 peptide and monkeys M88 and M91 receiving the mix of GP1+GP14. The results are expressed as a mean of SFCs, i.e. of colonies secreting Interferon-g in an Elispot assay. Results are strongly positive in all monkeys towards several peptides, e.g. the recombinant 729 and immunising peptides GP1 in M73, as well as P. falciparium sporozoites, most peptides or sporozoites employed in monkey V114, the recombinmits 729 and NN for monkey M8S and M91 as well as the immunising peptide GP1 and, to a lesser extent GP14 in the same monkeys, as well as P. falciparium sporozoites in the same monkeys.

FIGS. 8a-d: LDII levels

The figure shows the various levels of LDH detected in the various monkeys mentioned above, as compared to the controls (blue line). The horizontal line is the threshold of positivity determined as the mean OD value in controls+3 standard the assay determined as the mean value given by uninfected aotus control blood+3 standard deviations (results below this threshold value are negative and results above this threshold value are positive). The horizontal axis indicates the days following-sporozoite injection, when samples were taken and processed in the DELI-LDH assay.

### EXAMPLE 4

Sequence Data and Supplementary Information

The following further information exemplifying the invention is supplied:

Sequence Data—Gene: full Sequence (K1 parasite)

Protein: full Sequence (K1 parasite)

Clones DG729/DG679 (T9/96 parasite)

Note on LSA-3 sequence in parasite 3D7

Gene & Protein—Structure. Restriction map. Hydrophobicity

Oligonucleotides employed

Organisation

Regions & Comments—NR-A.R1.R2.NR-B.R3.NR-C

Conservation—of the gene

of the sequence

of repeat region R2

comparaison of immunising and challenging sequences

Stage Specificity & Subcellular Location

Homologies—Intraspecies

Interspecies

Synthetic Peptides & Recombinant Proteins used for Chimpanzee Immunisations

Peptides CT1.NR1.NR2.RE

Recombinant proteins B-729. GST-729. OST-NN. GST-PC

Methods

References to Example 4

Full sequence listings in the appropriate format are also provided herein.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1 A vaccine composition comprising a Th1-inducing adjuvant in combination with a protecting Liver Stage Antigen or immunological fragment thereof of a human malaria parasite with the proviso that when the immunological fragment is an immunological fragment of LSA-3, the Th1-inducing adjuvant is not Montanide.

2 A vaccine composition as claimed in claim 1 wherein the human malaria parasite is Plasmodium falciparum.

3 A vaccine composition as claimed in claim 1 or claim 2 in which the Th1-inducing adjuvant comprises either (a) QS21, De-O-acylated monophosphoryl lipid A (3D-MPL) and an oil in water emulsion wherein the oil in water emulsion has the following composition: a metabolisable oil, such a squalene, alpha tocopherol and tween 80; or (b) a vesicular adjuvant formulation comprising cholesterol, a saponin and optionally an LPS derivative.

4 A vaccine composition as claimed in claim 1 or 2 or claim 3 wherein said protecting Liver Stage Antigen is the Liver Stage Antigen 3 (LSA-3) or immunological fragment thereof.

5 A vaccine composition according to any one of claims 1 to 4 comprising in addition at least one other protecting antigen or an immunological fragment thereof, of a malaria parasite.

6 A vaccine composition as claimed in claim 4 in which the other malaria antigen is selected from the following group:
a) a hybrid protein comprising substantially all the C-terminal portion of the CS protein, four or more tandem repeats of the immunodominant region, and the surface antigen from hepatitis B virus (HBsAg), in particular RTS,S, or immunogenic derivatives including fragments thereof;
b) the TRAP protein of the T9/96 isolate of Plasmodium falciparum and proteins having at least 80% homology thereto and immunogenic derivatives including fragments thereof;
c) the MSP-1 of Plasmodium falciparum or Plasmodium vivax and proteins having at least 80% homology thereto and immunogenic derivatives including fragments thereof; and
d) the MSP-3 of Plasmodium falciparum or Plasmodium vivax and proteins having at least 70% homology with the C-terminal region thereof, and immunogenic derivatives including fragments thereof.

7 A vaccine composition according to claims 1 to 6 capable of involving a T cell response in a mammal to the antigen or antigenic composition

8 A vaccine composition according to claims 1 to 7 capable of stimulating interferon γ production.

9 A vaccine composition according to claims 1 to 8, wherein the ratio of QS21:3D-MPL is from 1:10 to 10:1.

10 A vaccine composition according to claims 1 to 8, wherein the ratio of QS21:3D-MPL is from 1:1 to 1:2.5.

11 A process to make a vaccine composition according to any one of claims 1 to 10 comprising admixing QS21, 3D-MPL and the oil in water emulsion as defined in claim 2 with a protecting Liver Stage Antigen of a human malaria parasite.

12 A process according to claim 11 wherein the Liver Stage Antigen is LSA-3 of Plasmodium falciparum or immunological fragment thereof.

13 Use of a composition according to any one of claims 1 to 10 for the prophylaxis or treatment of malaria infections.


SEQUENCE LISTING (10 sequences)

SEQ ID NO: 1 - DNA, 5528 bp, K1 Parasite Strain
atgacaaatagtaattacaaatcaaataataaaacatataatgaaaataataatgaacaaataactaccatatttaatagaacaaatatgaatccgataaaaaaatgtcatatgagagaaaaaataaataagtacttttttttgatcaaaattttgacatgcaccattttaatatgggctgtacaatatgataataacgtaagataaaaaactaaataataaatataaataaaaaaaaaaaaaaaaaaaaaaaaatcaactatatagtatgtataatatatatatatatatatatatatatatatatatatatatatttatttttatttatttattaatttttttttttttatattatctttttagtctgatataaacaagagttggaaaaaaaatacgtatgtagataagaaattgaataaactatttaacagaagtttaggagaatctcaagtaaatggtgaattagctagtgaagaagtaaaggaaaaaattcttgacttattagaagaaggaaatacattaactgaaagtgtagatgataataaaaatttagaagaagccgaagatataaaggaaaatatcttattaagtaatatagaagaaccaaaagaaaatattattgacaatttattaaataatattggacaaaattcagaaaaacaagaaagtgtatcagaaaatgtacaagtcagtgatgaactttttaatgaattattaaatagtgtagatgttaatggagaagtaaaagaaaatattttggaggaaagtcaagttaatgacgatatttttaatagtttagtaaaaagtgttcaacaagaacaacaacacaatgttgaagaaaaagttgaagaaagtgtagaagaaaatgacgaagaaagtgtagaagaaaatgtagaagaaaatgtagaagaaaatgacgacggaagtgtagcctcaagtgttgaagaaagtatagcttcaagtgttgatgaaagtatagattcaagtattgaagaaaatgtagctccaactgttgaagaaatcgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctgaaaatgttgaagaaagttagctgaaaatgttgaagaaatcgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaatcgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctccaactgttgaagaaattgtagctccaagtgttgaagaaagtgtagctccaagtgttgaagaaagtgttgctgaaaacgttgcaacaaatttatcagacaatcttttaagtaatttattaggtggtatcgaaactgaggaaataaaggacagtatattaaatgagatagaagaagtaaaagaaaatgtagtcaccacaatactagaaaacgtagaagaaactacagctgaaagtgtaactacttttagtaacatattagaggagatacaagaaaatactattactaatgatactatagaggaaaaattagaagaactccacgaaaatgtattaagtgccgctttagaaaatacccaaagtgaagaggaaaagaaagaagtaatagatgtaattgaagaagtaaaagaagaggtcgctaccactttaatagaaactgtggaacaggcagaagaaaagagcgcaaatacaattacggaaatatttgaaaatttagaagaaaatgcagtagaaagtaatgaaaatgttgcagagaatttagagaaattaaacgaaactgtatttaatactgtattagataaagtagaggaaacagtagaaattagcggagaaagtttagaaaacaatgaaatggataaagcattttttagtgaaatatttgataatgtaaaaggaatacaagaaaatttattaacaggtatgtttcgaagtatagaaaccagtatagtaatccaatcagaagaaaaggttgatttgaatgaaaatgtggttagttcgattttagataatatagaaaatatgaaagaaggtttattaaataaattagaaaatatttcaagtactgaaggtgttcaagaaactgtaactgaacatgtagaacaaaatgtatatgtggatgttgatgttcctgctatgaaagatcaatttttaggaatattaaatgaggcaggagggttgaaagaaatgttttttaatttggaagatgtatttaaaagtgaaagtgatgtaattactgtagaagaaattaaggatgaaccggttcaaaaagaggtagaaaaagaaactgttagtattattgaagaaatggaagaaaatattgtagatgtattagaggaagaaaaagaagatttaacagacaagatgatagatgcagtagaagaatccatagaaatatcttcagattctaaagaagaaactgaatctattaaagataaagaaaaagatgtttcactagttgttgaagaagttcaagacaatgatatggatgaaagtgttgagaaagttttagaattgaaaaatatggaagaggagttaatgaaggatgctgttgaaataaatgacattactagcaaacttattgaagaaactcaagagttaaatgaagtagaagcagatttaataaaagatatggaaaaattaaaagaattagaaaaagcattatcagaagattctaaagaaataatagatgcaaaagatgatacattagaaaaagttattgaagaggaacatgatataacgacgacgttggatgaagttgtagaattaaaagatgtcgaagaagacaagatcgaaaaagtatctgatttaaaagatcttgaagaagatatattaaaagaagtaaaagaaatcaaagaacttgaaagtgaaattttagaagattataaagaattaaaaactattgaaacagatattttagaagagaaaaaagaaatagaaaaagatcattttgaaaaattcgaagaagaagctgaagaaataaaagatcttgaagcagatatattaaaagaagtatcttcattagaagttgaagaagaaaaaaaattagaagaagtacacgaattaaaagaagaggtagaacatataataagtggtgatgcgcatataaaaggtttggaagaagatgatttagaagaagtagatgatttaaaaggaagtatattagacatgttaaagggagatatggaattaggggatatggataaggaaagtttagaagatgtaacaacaaaacttggagaaagagttgaatccttaaaagatgttttatctagtgcattaggcatggatgaagaacaaatgaaaacaagaaaaaaagctcaaagacctaagttggaagaagtattattaaaagaagaggttaaagaagaaccaaagaaaaaaataacaaaaaagaaagtaaggtttgatattaaggataaggaaccaaaagatgaaatagtagaagttgaaatgaaagatgaagatatagaagaagatgtagaagaagatatagaagaagatatagaagaagataaagttgaagatatagatgaagatatagatgaagatataggtgaagacaaagatgaagttatagatttaatagtccaaaaagagaaacgcattgaaaaggttaaagcgaaaaagaaaaaattagaaaaaaaagttgaagaaggtgttagtggtcttaaaaaacacgtagacgaagtaatgaaatatgttcaaaaaattgataaagaagttgataaagaagtatctaaagctttagaatcaaaaaatgatgttactaatgttttaaaacaaaatcaagatttttttagtaaagttaaaaacttcgtaaaaaaatataaagtatttgctgcaccattcatatctgccgttgcagcatttgcatcatatgtagttgggttctttacattttctttattttcatcatgtgtaacaatagcttcttcaacttacttattatcaaaagttgacaaaactataaataaaaataaggagagaccgttttattcatttgtatttgatatctttaagaatttaaaacattatttacaacaaatgaaagaaaaatttagtaaagaaaaaaataataatgtaatagaagtaacaaacaaagctgagaaaaaaggtaatgtacaggtaacaaataaaaccgagaaaacaactaaagttgataaaaataataaagtaccgaaaaaaagaagaacgcaaaaatcaaaataa

SEQ ID NO: 2 - protein, 1787 aa, K1 Parasite Clone
MTNSNYKSNNKTYNENNNEQITTIFNRTNMNPIKKCHMREKINKYFFLIKILTCTILIWAVQYDNNSDINKSWKKNTYVDKKLNKLFNRSLGESQVNGELASEEVKEKILDLLEEGNTLTESVDDNKNLEEAEDIKENILLSNIEEPKENIIDNLLNNIGQNSEKQESVSENVQVSDELFNELLNSVDVNGEVKENILEESQVNDDIFNSLVKSVQQEQQHNVEEKVEESVEENDEESVEENVEENVEENDDGSVASSVEESIASSVDESIDSSIEENVAPTVEEIVAPSVVESVAPSVEESVEENVEESVAENVEESVAENVEESVAENVEESVAENVEEIVAPTVEEIVAPTVEEIVAPSVVESVAPSVEESVEENVEESVAENVEESVAENVEESVAENVEESVAENVEESVAENVEEIVAPTVEEIVAPTVEEIVAPSVVESVAPSVEESVEENVEESVAENVEESVAENVEESVAENVEESVAENVEESVAENVEESVAENVEESVAENVEEIVAPTVEEIVAPTVEEIVAPSVVESVAPSVEESVEENVEESVAENVEESVAENVEESVAENVEESVAENVEEIVAPTVEEIVAPTVEEIVAPSVVESVAPSVEESVEENVEESVAENVEESVAENVEESVAENVEEIVAPTVEEIVAPTVEEIVAPSVVESVAPSVEESVEENVEESVAENVEESVAENVEESVAENVEESVAENVEEIVAPTVEEIVAPTVEEIVAPSVVESVAPSVEESVEENVEESVAENVEESVAENVEESVAENVEESVAPTVEEIVAPSVEESVAPSVEESVAENVATNLSDNLLSNLLGGIETEEIKDSILNEIEEVKENVVTTILENVEETTAESVTTFSNILEEIQENTITNDTIEEKLEELHENVLSAALENTQSEEEKKEVIDVIEEVKEEVATTLIETVEQAEEKSANTITEIFENLEENAVESNENVAENLEKLNETVFNTVLDKVEETVEISGESLENNEMDKAFFSEIFDNVKGIQENLLTGMFRSIETSIVIQSEEKVDLNENVVSSILDNIENMKEGLLNKLENISSTEGVQETVTEHVEQNVYVDVDVPAMKDQFLGILNEAGGLKEMFFNLEDVFKSESDVITVEEIKDEPVQKEVEKETVSIIEEMEENIVDVLEEEKEDLTDKMIDAVEESIEISSDSKEETESIKDKEKDVSLVVEEVQDNDMDESVEKVLELKNMEEELMKDAVEINDITSKLIEETQELNEVEADLIKDMEKLKELEKALSEDSKEIIDAKDDTLEKVIEEEHDITTTLDEVVELKDVEEDKIEKVSDLKDLEEDILKEVKEIKELESEILEDYKELKTIETDILEEKKEIEKDHFEKFEEEAEEIKDLEADILKEVSSLEVEEEKKLEEVHELKEEVEHIISGDAHIKGLEEDDLEEVDDLKGSILDMLKGDMELGDMDKESLEDVTTKLGERVESLKDVLSSALGMDEEQMKTRKKAQRPKLEEVLLKEEVKEEPKKKITKKKVRFDIKDKEPKDEIVEVEMKDEDIEEDVEEDIEEDIEEDKVEDIDEDIDEDIGEDKDEVIDLIVQKEKRIEKVKAKKKKLEKKVEEGVSGLKKHVDEVMKYVQKIDKEVDKEVSKALESKNDVTNVLKQNQDFFSKVKNFVKKYKVFAAPFISAVAAFASYVVGFFTFSLFSSCVTIASSTYLLSKVDKTINKNKERPFYSFVFDIFKNLKHYLQQMKEKFSKEKNNNVIEVTNKAEKKGNVQVTNKTEKTTKVDKNNKVPKKRRTQKSKZ

SEQ ID NO: 3 - DNA, 1712 bp, T9/96 Parasite Clone
agtgatgaactttttaatgaattattaaatagtgtagatgttaatggagaagtaaaagaaaatattttggaggaaagtcaagttaatgacgatatttttaatagtttagtaaaaagtgttcaacaagaacaacaacacaatgttgaagaaaaagttgaagaaagtgtagaagaaaatgacgaagaaagtgtagaagaaaatgtagaagaaaatgtagaagaaaatgacgacggaagtgtagcctcaagtgttgaagaaagtatagcttcaagtgttgatgaaagtatagattcaagtattgaagaaaatgtagctccaactgttgaagaaatcgtagctccaactgttgaagaaattgtagctccaagtgttgtagaaagtgtggctccaagtgttgaagaaagtgtagctccaagtgttgaagaaagtgtagctgaaaatgttgaagaaagtgtagctgaaaatgttgaagaaatcgtagctccaagtgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaactgttgaagaaagtgtagctccaactgttgaagaaattgtagctccaactgttgaagaaagtgtagctccaactgttgaagaaattgtagttccaagtgttgaagaaagtgtagctccaagtgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaagtgttgaagaaatcgtagctccaactgttgaagaaagtgttgctgaaaacgttgcaacaaatttatcagacaatcttttaagtaatttattaggtggtatcgaaactgaggaaataaaggacagtatattaaatgagatagaagaagtaaaagaaaatgtagtcaccacaatactagaaaaagtagaagaaactacagctgaaagtgtaactacttttagtaatatattagaggagatacaagaaaatactattactaatgatactatagaggaaaaattagaagaactccacgaaaatgtattaagtgccgctttagaaaatacccaaagtgaagaggaaaagaaagaagtaatagatgtaattgaagaagtaaaagaagaggtcgctaccactttaatagaaactgtggaacaggcagaagaagagagcgaaagtacaattacggaaatatttgaaaatttagaagaaaatgcagtagaaagtaatgaaaaagttgcagagaatttagagaaattaaacgaaactgtatttaatactgtattagataaagtagaggaaacagtagaaattagcggagaaagtttagaaaacaatgaaatggataaagcattttttagtgaaatatttgataatgtaaaaggaatacaagaaaatttattaacaggtatgtttcgaagtatagaaaccagtatagtaatccaatcagaagaaaaggttgatttgaatgaaaatgtggttagttcgattttagataatatagaaaatatgaaagaaggtttattaaataaattagaaaatatttcaagtactgaagg

SEQ ID NO: 4 - protein, 570 aa, T9/96 Parasite Clone
SDELFNELLNSVDVNGEVKENILEESQVNDDIFNSLVKSVQQEQQHNVEEKVEESVEENDEESVEENVEENVEENDDGSVASSVEESIASSVDESIDSSIEENVAPTVEEIVAPTVEEIVAPSVVESVAPSVEESVAPSVEESVAENVEESVAENVEEIVAPSVEESVAENVEESVAENVEESVAENVEESVAENVEESVAENVEEIVAPTVEESVAPTVEEIVAPTVEESVAPTVEEIVVPSVEESVAPSVEESVAENVEESVAENVEESVAENVEESVAENVEESVAENVEEIVAPSVEEIVAPTVEESVAENVATNLSDNLLSNLLGGIETEEIKDSILNEIEEVKENVVTTILEKVEETTAESVTTFSNILEEIQENTITNDTIEEKLEELHENVLSAALENTQSEEEKKEVIDVIEEVKEEVATTLIETVEQAEEESESTITEIFENLEENAVESNEKVAENLEKLNETVFNTVLDKVEETVEISGESLENNEMDKAFFSEIFDNVKGIQENLLTGMFRSIETSIVIQSEEKVDLNENVVSSILDNIENMKEGLLNKLENISSTE

SEQ ID NO: 5 - protein, 20 aa, Artificial Sequence
  Synthetic peptide
LLSNIEEPKENIIDNLLNNI

SEQ ID NO: 6 - protein, 25 aa, Artificial Sequence
  Synthetic peptide
DELFNELLNSVDVNGEVKENILEES

SEQ ID NO: 7 - protein, 26 aa, Artificial Sequence
  Synthetic peptide
LEESQVNDDIFNSLVKSVQQEQQHNV

SEQ ID NO: 8 - protein, 28 aa, Artificial Sequence
  Synthetic peptide
VESVAPSVEESVAPSVEESVAENVEESV

SEQ ID NO: 9 - protein, 123 aa, Artificial Sequence
  Synthetic peptide
LASEEVKEKILDLLEEGNTLTESVDDNKNLEEAEDIKENILLSNIEEPKENIIDNLLNNIGQNSEKQESVSENVQVSDELFNELLNSVDVNGEVKENILEESQVNDDIFNSLVKSVQQEQQHN

SEQ ID NO: 10 - protein, 96 aa, Artificial Sequence
  Synthetic peptide
ESVAENVEESVAENVEEIVAPTVEEIVAPTVEEIVAPSVVESVAPSVEESVEENVEESVAENVEESVAENVEESVAENVEESVAENVEEIVAPTVE